(19) United States
(12) Patent Application Publication
(10) Pub. No.: US 2026/0358452 A1
(43) Pub. Date: Nov. 12, 2026

(54) MAGNETIC RESONANCE APPARATUS WITH A RADIO FREQUENCY COIL CONFIGURED TO BE LOCKED IN A PREDETERMINED POSITION

(71) Applicant: Siemens Healthineers AG, Forchheim (DE)

(72) Inventor: Wolfgang Neuber, Eschenbach i. d. Opf. (DE)

(21) Appl. No.: 19/658,924

(22) Filed: Apr. 27, 2026

(30) Foreign Application Priority Data
May 9, 2025 (DE) 10 2025 117 876.3

(51) Int. Cl. G01R 33/34; A61B 5/055; G01R 33/30

(52) U.S. Cl. CPC G01R 33/34007; A61B 5/055; G01R 33/307

(57) ABSTRACT

A magnetic resonance apparatus includes: a patient table having a patient positioning surface and a radio frequency coil; a magnet unit surrounding a patient recording area; and a holding apparatus. The patient positioning surface and the radio frequency coil are configured to move together along an axis of displacement in a first section of the patient recording area. The holding apparatus is configured to hold the radio frequency coil in a position relative to the magnet unit when the patient positioning surface is moved along the axis of displacement in a second section. The holding apparatus includes a counterpart and a recording unit for recording the counterpart. The recording unit is arranged in a fixed manner relative to the magnet unit. The holding apparatus is configured to hold the counterpart in the recording unit when the patient positioning surface is located along the axis of displacement in the second section.

The present patent document claims the benefit of German Patent Application No. 10 2025 117 876.3, filed May 9, 2025, which is hereby incorporated by reference in its entirety.

### TECHNICAL FIELD

The disclosure relates to a magnetic resonance apparatus with a patient table and a magnet unit.

### BACKGROUND

In medical technology, imaging using magnetic resonance (MR), also referred to as magnetic resonance tomography (MRT) or magnetic resonance imaging (MRI), is characterized by high soft tissue contrast. In the process, a patient is positioned on a patient table in a patient recording or accommodation area of a magnetic resonance apparatus in which a main magnet of the magnetic resonance apparatus generates a main magnetic field. During a magnetic resonance measurement, one or more radio frequency coils are used to transmit radio frequency (RF) transmission pulses into the examination area according to a magnetic resonance sequence in order to generate an alternating magnetic field, thus exciting nuclear spins in the patient. As a result, magnetic resonance signals, which may be received by one or more radio frequency coils, are triggered in the patient. The magnetic resonance signals may be used for reconstructing magnetic resonance mappings.

One possible embodiment of radio frequency coils is a spine coil, sometimes also referred to as a back coil. Magnetic resonance apparatuses are known in which the spine coil is inserted into a recess provided for this purpose on the top of the patient table. The length of the spine coil determines the measurement range, so the spine coil may be as long as possible in order to be able to cover a large measurement range. However, the manufacture of a long spine coil also incurs high costs.

### SUMMARY AND DESCRIPTION

The object of the present disclosure may be regarded as specifying a magnetic resonance apparatus that efficiently enables a large posterior, in particular dorsal, measurement range.

The scope of the present disclosure is defined solely by the appended claims and is not affected to any degree by the statements within this summary. The present embodiments may obviate one or more of the drawbacks or limitations in the related art.

Accordingly, a magnetic resonance apparatus with a patient table and a magnet unit is proposed, the magnet unit surrounding a patient recording or accommodation area. The patient table has a patient positioning surface, in particular, for positioning a patient. Furthermore, the patient table includes a radio frequency coil. The magnetic resonance apparatus is configured in such a manner that the patient positioning surface and the radio frequency coil are moved together along an axis of displacement in a first section (e.g., first movement section), in particular simultaneously and/or in parallel. Furthermore, the magnetic resonance apparatus includes a holding apparatus configured to hold the radio frequency coil in a predefined position relative to the magnet unit, in particular to lock and/or secure the radio frequency coil there, e.g., when the patient positioning surface is moved (further) along the axis of displacement in the second section (e.g., second movement section). Advantageously, as a result, the radio frequency coil is held in the predefined position relative to the magnet unit, i.e., the holding apparatus is configured to hold the counterpart in the recording unit so that the radio frequency coil is held in the predefined position relative to the magnet unit.

The magnetic resonance apparatus may be configured in such a manner that the patient positioning surface and the radio frequency coil are moved together along the axis of displacement in the first section in the patient recording area of the magnetic resonance apparatus. The patient positioning surface and the radio frequency coil may be configured in such a manner that the patient positioning surface is moved further along the axis of displacement in the second section, while the radio frequency coil remains stationary. The second section may immediately follow the first section.

The radio frequency coil may be a spine coil. The holding apparatus includes a counterpart and a recording unit for recording the counterpart. The recording unit is arranged in a fixed, in particular immovable, manner relative to the magnet unit, the counterpart being connected to the radio frequency coil. The holding apparatus is configured to hold the counterpart in the recording unit when the patient positioning surface is located along the axis of displacement in the second section.

Advantageously, the radio frequency coil may be locked in the predefined position. The predefined position may be a measurement position. The predefined position may be a position suitable for performing a magnetic resonance measurement with the radio frequency coil arranged in the predefined position. In the predefined position, the radio frequency coil may be located in a measurement field in which the main magnetic field of the magnetic resonance apparatus exhibits a sufficiently high degree of homogeneity. The predefined position may be a position of the radio frequency coil that is suitable for transmitting and/or receiving radio frequency signals for a magnetic resonance measurement using the radio frequency coil. Advantageously, in the predefined position, the radio frequency coil covers a reception area for receiving magnetic resonance signals. The predefined position may be located in the vicinity of the part of the body of a patient to be measured and/or in the vicinity of a field of view (FOV) of a magnetic resonance measurement and/or in the vicinity of the isocenter of the magnetic resonance apparatus. Advantageously, the distance between the field of view and the radio frequency coil and/or between the isocenter and the radio frequency coil is minimal when the radio frequency coil is located in the predefined position along the axis of displacement. For example, the predefined position along the axis of displacement is located in the center of the magnet unit. Advantageously, magnetic resonance signals with a high signal-to-noise ratio may be acquired with the radio frequency coil when the radio frequency coil is located in the predefined position.

By holding the counterpart in the recording unit, the radio frequency coil is advantageously held in the predefined position relative to the magnet unit, in particular temporarily fixed. By holding the counterpart in the recording unit, the radio frequency coil is advantageously located in the predefined position relative to the magnet unit. Advantageously, holding the radio frequency coil in the predefined position involves securing and/or locking the radio frequency coil in the predefined position. Advantageously, holding the radio frequency coil in the predefined position prevents movement of the radio frequency coil relative to the magnet unit.

By holding the counterpart in the recording unit, the radio frequency coil may be held close to the field of view and/or close to the isocenter of the magnetic resonance apparatus. The radio frequency coil may be moved along the axis of displacement in the first movement section up to the level of a field of view of a magnetic resonance measurement and/or an isocenter of the magnetic resonance apparatus and is then held there. Advantageously, by holding the radio frequency coil in the predefined position, a relatively large (e.g., posterior; located below the patient) measurement range along the axis of displacement may be covered by a relatively short radio frequency coil. The size of the measurement range depends in particular on the length of the second movement section in which the radio frequency coil is held in the predefined position.

The patient table may be part of a patient positioning apparatus that, in addition to the patient table, may include a base unit, (e.g., a frame and/or a support structure and/or a pedestal), on which the patient table is supported and/or mounted. The magnetic resonance apparatus, (e.g., the patient positioning apparatus), may include a motor, (e.g., an electric motor), to perform the movement, in particular the displacement, of the patient positioning surface along the axis of displacement. The displacement, (e.g., the motorized displacement), may be performed by operating personnel of the magnetic resonance apparatus.

The magnet unit may include a main magnet, (e.g., a superconducting main magnet), for generating a main magnetic field and/or a gradient coil unit for generating magnetic field gradients. The main magnetic field may have a strength in a range of 0.5 to 12 tesla. Furthermore, the magnet unit may include a body coil, e.g., for transmitting and/or receiving radio frequency signals. The main magnet and/or the gradient coil unit and/or the body coil may be permanently installed in the magnet unit.

The patient recording area may be configured as a bore in the magnet unit. The patient recording area may be configured as a hollow space in the magnet unit. The patient recording area may be cylindrical, i.e., have the shape of a cylinder, e.g., a circular cylinder. The central axis of such a cylinder may be aligned parallel to the axis of displacement. The patient recording area may be delimited in a circumferential direction by a housing wall of the magnet unit. The patient recording area may be delimited in a longitudinal direction by openings in a bore of the magnet unit characterizing the patient recording area. Advantageously, the patient may be brought into the patient recording area through the openings.

The radio frequency coil may be configured for transmitting and/or receiving radio frequency signals. The radio frequency coil may be configured for transmitting excitation pulses of a magnetic resonance sequence and/or for receiving magnetic resonance signals. The radio frequency coil may include one or more coil elements. A coil element may include a loop-shaped radio frequency antenna. The radio frequency coil may be a spine coil. The radio frequency coil may have the shape of a flat, in particular rectangular, plate. The radio frequency coil may have a length (e.g., parallel to the axis of displacement), a width (e.g., perpendicular to the axis of displacement), and a thickness (e.g., perpendicular to the axis of displacement). The length and the width may be significantly greater than the thickness, in particular, more than five times or more than ten times as great.

The patient positioning surface may be configured as a table surface. The patient positioning surface may be configured to support a patient. The radio frequency coil may be arranged under the patient positioning surface. In particular, the patient positioning surface is located between a patient and the radio frequency coil during a magnetic resonance measurement. The radio frequency coil may have a main surface aligned parallel to the patient positioning surface. The dimensions of the main surface of the radio frequency coil may be determined by the length and width of the radio frequency coil. Advantageously, the distance between the radio frequency coil and a patient lying on the patient positioning surface is kept as short as possible in order to achieve a high signal-to-noise ratio for the received magnetic resonance signals. The radio frequency coil may be integrated into the patient table and/or installed in a fixed (but moveable therein) and/or permanent manner. The patient table may include a housing that surrounds the radio frequency coil.

The first section of the movement of the patient positioning surface may relate to a movement by which the patient table, e.g., a patient lying thereon, is moved from the outside into the patient recording area through a first opening. The second section of the movement of the patient positioning surface may relate to a movement by which the patient table, in particular the patient lying thereon, is moved past a predetermined point. The patient table may be moved along the axis of displacement out of the patient recording area again through an opening opposite the first opening by this movement.

In particular, the recording unit of the holding apparatus is arranged in an immovable manner relative to the magnet unit. In particular, the recording unit is arranged in a fixed manner relative to the magnet unit. In particular, the recording unit is attached to the magnet unit and/or securely connected to the magnet unit. The recording unit may be attached to the magnet unit by screws.

Advantageously, the counterpart of the holding apparatus moves with the radio frequency coil, or the radio frequency coil remains stationary when the counterpart also remains stationary, in particular is held in the recording unit. Advantageously, the movements of the patient positioning surface and the radio frequency coil are decoupled as long as the counterpart is held in the recording unit.

In a conventional arrangement, the total length of a spine coil may not be used for a single MR image recording. Advantageously, the part of the body of the patient to be measured is located at the isocenter of the magnetic resonance apparatus as the homogeneity of the main magnetic field is particularly high there. Advantageously, the radio frequency coil, in particular the spine coil, of the proposed magnetic resonance apparatus is only as large as necessary, in particular not larger than an area that may be used for receiving magnetic resonance signals. A smaller coil is advantageously less expensive to produce than a large coil.

Advantageously, the radio frequency coil is moved into the predefined position and held in position there by the holding apparatus. In particular, the radio frequency coil is accommodated in the patient table. The radio frequency coil may be positioned in a forward position, in particular as far forward as possible, in the patient table before the patient is moved onto it. When the radio frequency coil reaches the predefined position through movement in the first movement section, the radio frequency coil advantageously remains there, being held there by the holding apparatus. During further longitudinal travel of the patient positioning surface along the axis of displacement, the radio frequency coil remains stationary in the second movement section. In this second movement section, there is thus a relative movement of the patient positioning surface and the radio frequency coil.

The radio frequency coil may remain in this predefined position, and only when the patient positioning surface reaches the maximum possible scanning position of the radio frequency coil during extension does the patient positioning surface, together with the radio frequency coil, leave the bore of the magnet again until it reaches, for example, a longitudinal end position, in particular a transfer position, of the patient table, e.g., on a frame of the patient positioning apparatus.

Advantageously, holding, in particular securing and/or locking, of the radio frequency coil in the predefined position takes place without active intervention by operating personnel of the magnetic resonance apparatus and/or without the use of an actuator, for example, an electric or pneumatic actuator, controlled by the magnetic resonance apparatus. Advantageously, switching or monitoring signals in the magnetic resonance apparatus, (e.g., in the patient table), which may lead to malfunctions, may be avoided as a result.

Advantageously, holding, in particular locking, of the radio frequency coil in the predefined position takes place independently. In particular, the lock is not released until the patient table leaves the patient recording area again.

Advantageously, the fixed arrangement of the recording unit relative to the magnet unit enables particularly reliable positioning of the radio frequency coil in the predefined position. In contrast, for example, an arrangement of a recording unit on a movable patient positioning apparatus that may be detached from the magnetic resonance apparatus would be significantly less reliable due to the much greater mechanical tolerances and the risk of misalignment of the patient positioning apparatus.

The recording unit may be arranged in the patient recording area. In particular, the recording unit is arranged in a fixed manner in the magnet unit. For example, the recording unit is arranged on, and in particular attached to, a housing wall of the magnet unit, in particular an inner housing wall and/or a housing wall that borders the patient recording area. For example, the recording unit is arranged on, and in particular attached to, a body coil of the magnet unit.

Advantageously, the arrangement of the recording unit in the patient recording area of the magnet unit does not necessitate particularly high requirements with regard to the position tolerance of the recording unit. In particular, even a rough adjustment may be sufficient to provide the robust function of the holding apparatus.

Advantageously, by arranging the recording unit in the patient recording area of the magnet unit, a technical solution that is independent of the specific magnet unit may be provided. This may be advantageous when an operator uses multiple magnetic resonance apparatuses, each with mobile patient positioning apparatuses. In this case, full compatibility of the mobile patient positioning apparatuses with the magnetic resonance apparatuses may be enabled.

Advantageously, by arranging the recording unit in the patient recording area, the holding apparatus may be mounted close to the predefined position in which the radio frequency coil is held. As a result, in particular a precise positioning of the radio frequency coil in the predefined position may be made significantly easier and/or better.

Another embodiment of the magnetic resonance apparatus provides that the counterpart includes an insertion element, the holding apparatus being configured to insert the insertion element into the recording unit in order to hold the counterpart in the recording unit. The insertion element may be clamped and/or locked in the recording unit for this purpose.

Advantageously, the insertion element and the recording unit are configured to form a positive fit when the insertion element is inserted into the recording unit. Advantageously, the recording unit blocks, in particular locks, the movement of the insertion element in at least one direction, in particular in a direction parallel to the axis of displacement, when the insertion element is inserted into the recording unit. Advantageously, such a block, in particular a locking mechanism, may provide a particularly simple but nevertheless reliable mechanical securing and/or locking mechanism.

The insertion element may have the shape of a cylinder, in particular of a circular cylinder. The insertion element may be configured as a pin and/or a bolt. The insertion element may be a protruding component. A cylindrical component may advantageously be inserted into a recording unit particularly easily.

Another embodiment of the magnetic resonance apparatus provides that the recording unit includes a recess, the holding apparatus being configured to insert the insertion element into the recess in order to hold the counterpart in the recording unit. The recess may be a recess and/or a groove and/or a notch.

The recess may have an elongated and/or curved shape. The recess may function as a hook into which the insertion element may be hooked in order to hold the counterpart in the recording unit.

The shape of the recess may be U-shaped. The recess may have an opening through which the insertion element, which may be cylindrical, may be inserted into the recess. In certain examples, the recess may have an edge, (e.g., an internal edge), which is aligned perpendicular to the axis of displacement in at least one section of the recess. Advantageously, the edge blocks the movement of the insertion element, and thus also of the counterpart, parallel to the axis of displacement.

Another embodiment of the magnetic resonance apparatus provides that the holding apparatus includes a guide rail arranged in a fixed manner relative to the patient positioning surface, the counterpart including a contour sensor that contacts the guide rail. The guide rail may include a rail contour suitable for inserting the insertion element into the recording unit, in particular into the recess, and/or removing it therefrom.

Advantageously, the predefined position in which the radio frequency coil is held may be easily adjusted by adjusting the position of the guide rail. Advantageously, any reverse play or loose positioning of the radio frequency coil in the patient table may be reduced, and in particular eliminated, by adjustment.

The contour sensor may be an element (e.g., a roller) that rolls on the guide rail and/or a sliding element (e.g., a slider). Advantageously, a roller used as a contour sensor shows only minimal wear. Advantageously, a slider used as a contour sensor is particularly easy and/or space-saving to integrate into the holding apparatus.

Another embodiment of the magnetic resonance apparatus provides that the insertion element follows the movement of the contour sensor on the guide rail, this movement being determined by the rail contour of the guide rail and being transmitted to the insertion element via the contour sensor.

A force may be transmitted to the insertion element via the contour sensor, which is suitable for inserting the insertion element into the recording unit, in particular into the recess, and/or for removing it therefrom. The direction of this force may be predefined by the rail contour. The direction of the force may be dependent on the position of the contour sensor on the axis of displacement. The rail contour may change along with the axis of displacement, this change leading to a change in the direction of the force.

The rail contour may have a first rail section aligned parallel to the axis of displacement and a second rail section aligned parallel to the axis of displacement, the contour sensor contacting the first rail section in the first movement section and contacting the second rail section in the second movement section. The first rail section is connected to the second rail section via a slope, the insertion element being guided via the slope along a movement trajectory leading into and/or out of the recording unit, the movement trajectory being transmitted from the guide rail to the insertion element via the contour sensor.

Advantageously, the targeted insertion and removal of the insertion element into and from the recording unit by the guide rail may prevent increased mechanical stress on the involved components and/or interfaces during locking and unlocking.

Another embodiment of the magnetic resonance apparatus provides that the counterpart includes a spring, in particular a leaf spring, which is configured to press the contour sensor against the guide rail. Advantageously, the contour sensor continuously contacts the guide rail as a result.

Another embodiment of the magnetic resonance apparatus provides that the contour sensor is arranged coaxially with the insertion element. For example, the contour sensor is configured as a roller that has a roller axis, the roller axis simultaneously being part of the insertion element.

Another embodiment of the magnetic resonance apparatus provides that the counterpart is mounted so as to be pivotable relative to the recording unit. Advantageously, by a degree of rotational freedom of the counterpart, the counterpart, in particular the insertion element of the counterpart, may be inserted into and/or removed from the recording unit.

Another embodiment of the magnetic resonance apparatus provides that the counterpart is rotatably mounted about a rotational axis, the rotational axis being connected to the radio frequency coil. Advantageously, this enables the simple and/or space-saving connection of the counterpart to the radio frequency unit.

Another embodiment of the magnetic resonance apparatus provides that the counterpart includes a rotatably mounted pivot lever, the insertion element and the contour sensor being arranged on the pivot lever. Advantageously, the rotatably mounted pivot lever enables the contour sensor to follow the rail contour of the guide rail, in particular with particular flexibility.

The holding apparatus may contain no metal, e.g., no component made of metal. Advantageously, a metal-free design of the holding apparatus causes no artifacts in the MR imaging.

Further advantages, features, and details of the disclosure emerge from the embodiments described hereinafter, and from the drawings. Corresponding parts are labeled with the same reference characters in all the figures.

### BRIEF DESCRIPTION OF THE DRAWINGS

FIG. 1 depicts an example of a magnetic resonance apparatus with a patient table in an initial position or a final position for transferring a patient onto a patient positioning surface.

FIG. 2 depicts an example of a magnetic resonance apparatus with a patient table in a locking position after a joint forward movement of the patient positioning surface and the radio frequency coil in a first movement section.

FIG. 3 depicts an example of a magnetic resonance apparatus with a patient table in a position after a further forward movement of the patient positioning surface while the radio frequency coil is locked in a second movement section.

FIG. 4 depicts an example of a magnetic resonance apparatus with a patient table in an unlocked position after a backward movement of the patient positioning surface while the radio frequency coil is locked in the second movement section.

FIG. 5 depicts an example of a holding apparatus in a state in the initial position or final position, as well as during the combined forward movement of the patient positioning surface and the radio frequency coil in the first movement section.

FIG. 6 depicts an example of a holding apparatus in a state in the locked position or the unlocked position during locking or unlocking.

FIG. 7 depicts an example of a holding apparatus in a state during the continued forward movement of the patient positioning surface while the radio frequency coil is locked in the second movement section.

### DETAILED DESCRIPTION

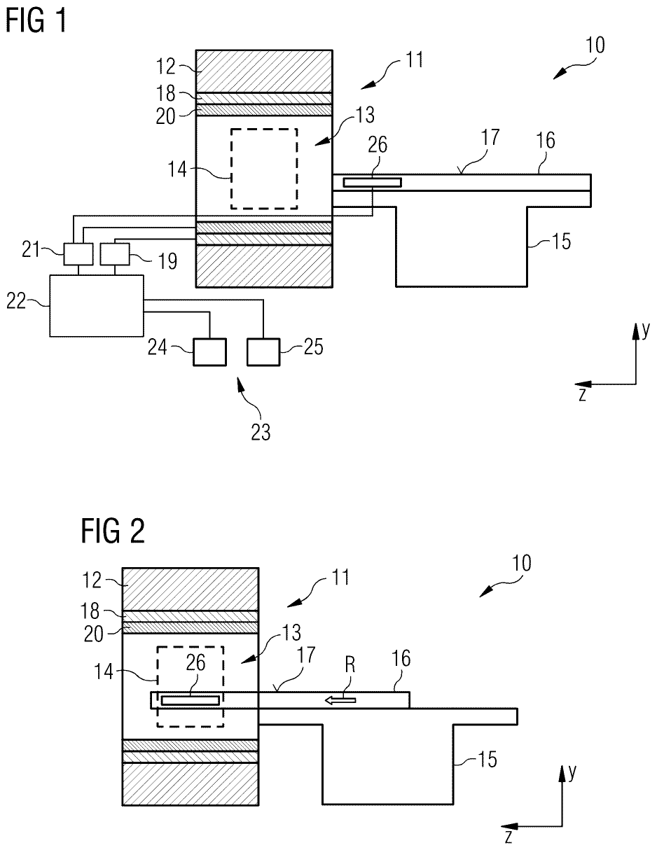

FIG. 1 schematically shows a magnetic resonance apparatus 10. The magnetic resonance apparatus 10 includes a magnet unit 11 that has a main magnet 12 for generating a strong and, in particular, temporally constant main magnetic field. In addition, the magnetic resonance apparatus 10 includes a patient recording or accommodation area 13 for recording a patient. The patient recording area 13 in the present embodiment is cylindrical in design and is enclosed in a cylindrical shape by the magnet unit 11 in a circumferential direction. The patient recording area 13 is configured as a bore in the magnet unit. The magnetic resonance apparatus 10 includes a patient positioning apparatus with a base unit 15 and a patient table 16 arranged thereon. The patient table 16 has a patient positioning surface 17 on which a patient may be placed. The patient table 16 may be moved into and out of the patient recording area 13 along an axis of displacement parallel to the z-axis. The part of the patient from which a magnetic resonance mapping is to be generated may be brought into a measurement field 14. The measurement field 14 may be located in the center of the magnet unit 11. The measurement field 14 is characterized in that the main magnetic field therein is particularly homogenous. A high degree of homogeneity in the main magnetic field is particularly necessary to provide high spatial resolution of the magnetic resonance signals. The field of view (FOV) of the magnetic resonance measurement may be in the measurement field 14. The measurement field may include the isocenter of the magnetic resonance apparatus 10.

The magnet unit 11 also has a gradient coil unit 18 for generating magnetic field gradients that are used for spatial encoding during imaging. The gradient coil unit 18 is controlled by a gradient coil unit 19 of the magnetic resonance apparatus 10. The magnet unit 11 further includes a body coil 20 that is permanently integrated into the magnetic resonance apparatus 10. The body coil 20 is controlled by a radio frequency antenna control unit 21 of the magnetic resonance apparatus 10 and emits radio frequency magnetic resonance sequences into an examination area that is predominantly formed by the patient recording area 13, in particular the measurement field 14, of the magnetic resonance apparatus 10. This results in the excitation of atomic nuclei by the main magnetic field generated by the main magnet 12. Magnetic resonance signals are generated by the relaxation of the excited atomic nuclei.

The magnetic resonance apparatus 10 further includes a radio frequency coil 26, which is here configured as a spine coil for receiving the magnetic resonance signals. The radio frequency coil 26 is arranged in the patient table 16. It is located below the patient positioning surface 17. The radio frequency coil 26 has a thickness in the y-direction that is significantly smaller (in particular by at least a factor of 5 or by at least a factor of 10) than its length in the z-direction. The width of the radio frequency coil 26 would then be its dimension perpendicular to the y-and z-direction, i.e., perpendicular to the drawing plane as viewed from the front.

The magnetic resonance apparatus 10 has a system control unit 22 for controlling the main magnet 12 and the gradient coil unit 19, and for controlling the radio frequency antenna control unit 21. The system control unit 22 centrally controls the magnetic resonance apparatus 10, for example by performing a predetermined magnetic resonance sequence. In addition, the system control unit 22 includes an evaluation unit, not shown in detail, for evaluating the magnetic resonance signals that are acquired during the magnetic resonance examination.

In addition, the magnetic resonance apparatus 10 includes a user interface 23 that is connected to the system control unit 22. Control information such as, for example, imaging parameters, as well as reconstructed magnetic resonance mappings may be displayed on a display unit 24, (e.g., on at least one monitor), of the user interface 23 for medical operating personnel. Furthermore, the user interface 23 has an input unit 25 by which information and/or parameters may be entered by the medical operating personnel during a measurement process.

FIGS. 1 to 4 show that the patient positioning surface 17 and the radio frequency coil 26 are configured to be moved together along an axis of displacement in a first section in the patient recording area 13 of the magnetic resonance apparatus 10, the magnetic resonance apparatus 10 including a holding apparatus configured to hold the radio frequency coil 26 in a predefined position relative to the magnet unit 11 when the patient positioning surface 17 is moved in a second section along the axis of displacement. To increase clarity, some components shown in FIG. 1 have been omitted in FIGS. 2 to 4.

A possible embodiment of the holding apparatus 27 is shown in FIGS. 5 to 7. The holding apparatus 27 includes a counterpart 29 and a recording unit 28 for recording the counterpart 29. The recording unit 28 is arranged in a fixed manner relative to the magnet unit 11. The counterpart 29 is connected to the radio frequency coil 26. The holding apparatus 27 is configured to hold the counterpart 29 in the recording unit 28 when the patient positioning surface 17 is located in the second section along the axis of displacement.

In FIG. 1, the patient table 16 is in a state in which the patient table 16 is retracted to its maximum extent. In this state, a patient may be positioned on the patient positioning surface 17 with particular ease. The position of the patient table 16 shown may therefore also be characterized as the patient transfer position. In this position, the radio frequency coil 26 is in the most forward position in the patient table 16. The distance between the radio frequency coil 26 and the end face of the patient table 16 closest to the magnet is minimal.

When the patient table 16 is moved through the right-hand opening of the bore into the patient recording area 13, the patient table 16 reaches the position shown in FIG. 2 after a certain distance. In this first section of the movement, the radio frequency coil 26 is moved together with the patient positioning surface 17 in the direction R along the axis of displacement (parallel to the z-axis), i.e., the relative position of the radio frequency coil 26 and the patient positioning surface 17 does not change during this process.

The radio frequency coil 26 is now located in the predefined position relative to the magnet unit 11, which is characterized here in that the radio frequency coil 26 is located in the measurement field 14 of the magnetic resonance apparatus 10. This position may represent the start of the scanning area of the patient table 16.

For example, the patient is positioned on the patient positioning surface 17 such that the head of the patient is located in the front area of the patient positioning surface 17, i.e., in the state shown in FIG. 2, the radio frequency coil 26 is located directly under the head of the patient. Thus, in this state, magnetic resonance signals for generating an MR image of the head of the patient may be recorded. In this position, the radio frequency coil 26 is locked in place by the holding apparatus 27.

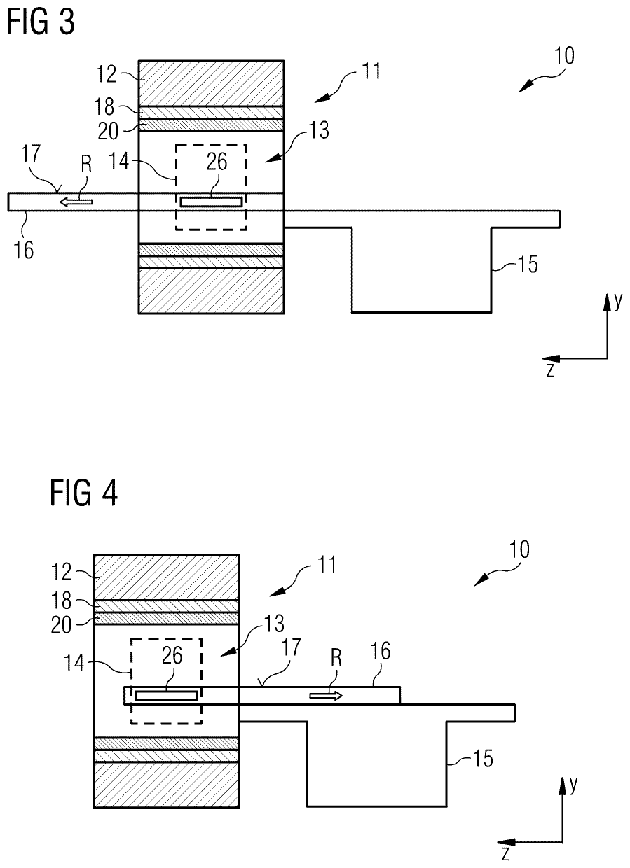

When the patient positioning surface 17 (together with the patient) is moved further along the axis of displacement in the direction R in this second section of the movement, the locking mechanism provided by the holding apparatus 27 provides that the radio frequency coil 26 remains stationary relative to the magnet unit 11. Conversely, the patient positioning surface 17 reaches the position shown in FIG. 3. This position may represent the end of the scanning area of the patient table 16. Advantageously, a relatively large scanning area may be covered by only a relatively short radio frequency coil 26 as the position of the patient positioned on the patient positioning surface 17 is variable relative to the radio frequency coil 26.

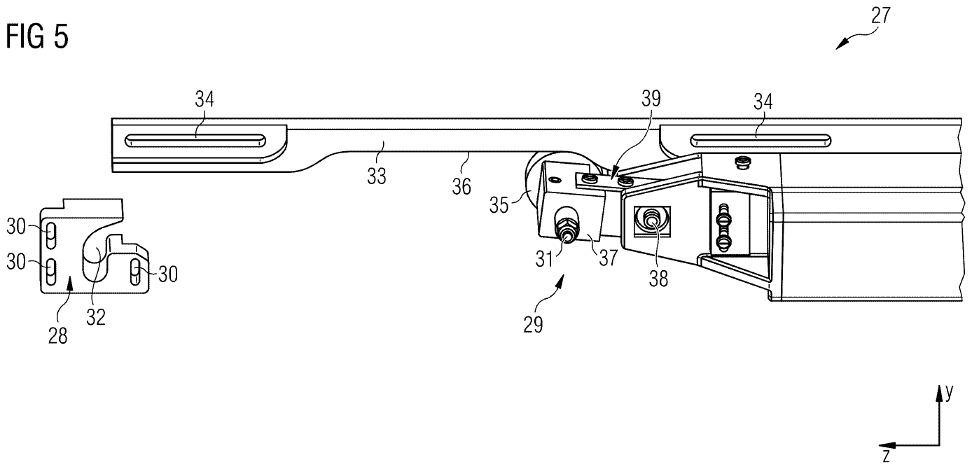

When the patient positioning surface 17 (together with the patient) is moved back along the axis of displacement, but now in the opposite direction R, the locking mechanism provided by the holding apparatus initially provides that the radio frequency coil 26 remains stationary relative to the magnet unit 11. Only when the patient positioning surface 17 reaches the position shown in FIG. 4 is the radio frequency coil 26 unlocked again, i.e., the radio frequency coil 26 is no longer held in position relative to the magnet unit 11. After further retraction of the patient table 16, the patient positioning surface 17, together with the radio frequency coil 26, then reaches the position shown in FIG. 1 again.

A possible holding apparatus 27 for locking the radio frequency coil 26 is explained in detail with the aid of FIGS. 5 to 7. The holding apparatus 27 may be located on the underside of the patient positioning surface 17, in particular in an area not visible from the outside.

The holding apparatus 27 includes a counterpart 29 and a recording unit 28 for recording the counterpart. The recording unit 28 is configured as a hook or functions as a hook. The recording unit 28 is arranged in a fixed manner relative to the magnet unit 11. In particular, the recording unit 28 is arranged in the patient recording area 13. The recording unit 28 may be attached to the inner surface of the bore of the magnetic resonance apparatus 10. The recording unit 28 has several slots 30 through which, for example, fastening screws may be inserted.

The counterpart 29 includes an insertion element 31 that may be inserted into the recording unit 28 in order to hold the counterpart 29 in the recording unit 28. The insertion element 31 is configured here as a cylindrical bushing.

The recording unit 28 has a recess 32 into which the insertion element 31 may be inserted in order to hold the counterpart 29 in the recording unit 28. The recess 32 has an elongated and curved shape.

The holding apparatus 27 also includes a guide rail 33 arranged in a fixed manner relative to the patient positioning surface 17. In particular, the guide rail 33 has slots 34 by which the guide rail 33 may be secured so that the guide rail 33 is immovable relative to the patient positioning surface 17. The counterpart 29 includes a contour sensor 35 in the form of a (switch) roller that contacts the guide rail 33. The insertion element 31 is mounted on the axis of the roller such that the roller is arranged coaxially with the insertion element 31. In particular, the insertion element 31 includes a bushing that is rotatable on this roller axis.

The guide rail 33 has a rail contour 36 suitable for inserting the insertion element 31 into the recording unit 28, in particular into the recess 32, and/or for removing the insertion element 31 therefrom. Furthermore, the counterpart 29 includes a spring 39 in the shape of a leaf spring that presses the contour sensor 35 against the guide rail 33.

The counterpart 29 includes a pivot lever 37 rotatably mounted on a rotational axis 38 of the holding apparatus 27 relative to the recording unit 28. The rotational axis 38 is in turn (mechanically) attached to the radio frequency coil 26 (not shown in FIGS. 5 to 7). The insertion element 31 and the contour sensor 35 are arranged on the pivot lever 37. In particular, the contour sensor 35 is rotatably mounted in the pivot lever 37. In particular, the spring 39 presses the pivot lever 37 upward in the direction of the patient positioning surface 17.

FIG. 5 shows the holding apparatus 27 in the state in which it is when the patient table 16 is in the position shown in FIG. 1. The slope of the rail contour 36 holds the radio frequency coil 26.

The holding apparatus 27 according to FIGS. 5 to 7 implements a switch roll mechanism attached to the radio frequency coil 26, which is configured to lock the radio frequency coil 26 into the recording unit 28 attached there when the radio frequency coil 26 reaches the measurement field 14, and to hold it stationary or locked there until the radio frequency coil 26 leaves the measurement field 14.

To enable longitudinal movement of the radio frequency coil 26 inside the patient table 16, the underside of the patient table 16 may have a longitudinal slot through which the rotational axis 38 extends. This allows the pivot lever 37 to move longitudinally within the defined area in the patient table 16 at the same time as the radio frequency coil 26.

In addition to the longitudinal slot in the patient table 16, in particular the guide rail 33 is attached there, which has a slanting, in particular chamfered, contour at its head end and then extends with a uniform width to the end of the longitudinal slot. Due to the spring 39, which pushes the pivot lever 37 upward, the contour sensor 35 is in contact with the guide rail 33.

As shown in FIG. 5, the spring 39 pushes the contour sensor 35 upward in the chamfered position and thus holds the radio frequency coil 26 in its end position (head-side end position in the patient table 16).

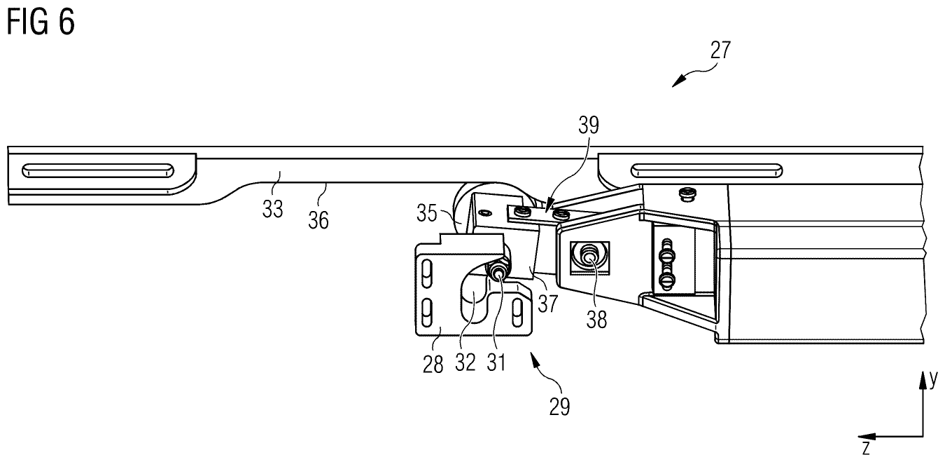

When, on retraction of the patient table 16, in particular the patient positioning surface 17, into the patient recording area 14, the counterpart 29, in particular the insertion element 31 that reaches the recording unit 28 that is fixed in the patient recording area 14, strikes it there and may no longer moves further along the z-axis longitudinally. However, the patient positioning surface 17 with the guide rail 33 continues to move, and the slanted rail contour pushes the contour sensor 35 downward (in the-y direction) with a movement component perpendicular to the axis of displacement, as shown in FIG. 6. As a result, the insertion element 31 moves into the U-shaped recess 32 of the recording unit 28.

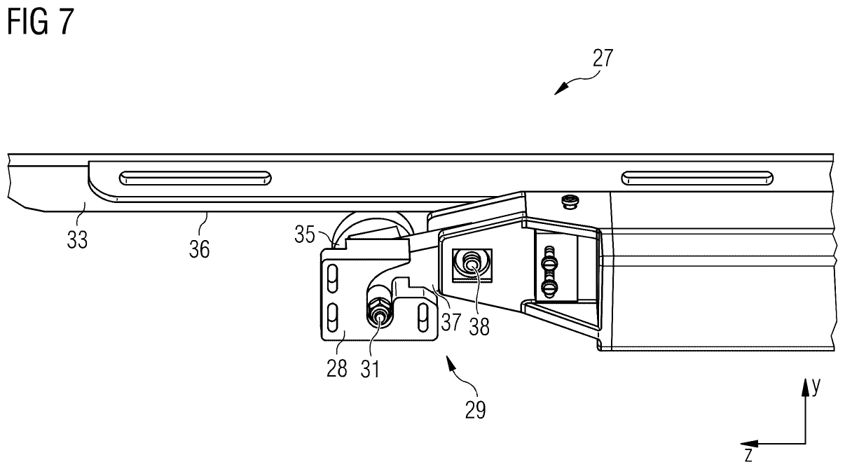

As shown in FIG. 7, as the patient positioning surface 17 continues to move longitudinally along the axis of displacement, the straight contour of the guide rail 33 holds the insertion element 31 in place in the recording unit 28, so that the radio frequency coil 26 remains locked in the predefined position in the measurement field.

The position of the radio frequency coil 26 is not unlocked or released again until the patient positioning surface 17 is moved out of the patient recording area 13 again and the slanted contour of the guide rail 33 allows the contour sensor 35 to swing out. The radio frequency coil 26 is then located in the initial position in the patient table 16 again and the patient table 16 may leave the patient recording area 13 and retract to a transfer position on the base unit 15 of the patient positioning apparatus.

Finally, the magnetic resonance apparatus 10 described in detail above is merely an embodiment that may be modified in various ways by a person skilled in the art without departing from the scope of the disclosure. Furthermore, the use of the indefinite article “a” or “an” does not preclude the possibility that the relevant features may be present in multiple instances. Likewise, the term “unit” does not preclude the possibility that the relevant components includes multiple interacting subcomponents, which possibly may also be spatially distributed.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A magnetic resonance apparatus comprising:
a patient table comprising a patient positioning surface and a radio frequency coil;
a magnet unit that surrounds a patient recording area; and
a holding apparatus comprising a counterpart and a recording unit arranged in a fixed manner relative to the magnet unit,
wherein the patient positioning surface and the radio frequency coil of the patient table are configured to move together along an axis of displacement in a first section of the patient recording area,
wherein the holding apparatus is configured to hold the radio frequency coil in a predefined position relative to the magnet unit when the patient positioning surface is moved along the axis of displacement in a second section of the patient recording area,
wherein the recording unit is configured to record the counterpart,
wherein the counterpart is connected to the radio frequency coil, and
wherein the holding apparatus is configured to hold the counterpart in the recording unit.

2. The magnetic resonance apparatus of claim 1, wherein the recording unit is arranged in the patient recording area.

3. The magnetic resonance apparatus of claim 1, wherein the counterpart comprises an insertion element,
wherein the holding apparatus is configured to insert the insertion element into the recording unit in order to hold the counterpart in the recording unit.

4. The magnetic resonance apparatus of claim 3, wherein the insertion element has a cylindrical shape.

5. The magnetic resonance apparatus of claim 3, wherein the recording unit comprises a recess, and
wherein the holding apparatus is configured to insert the insertion element into the recess in order to hold the counterpart in the recording unit.

6. The magnetic resonance apparatus of claim 5, wherein the recess has an elongated and curved shape.

7. The magnetic resonance apparatus of claim 3, wherein the holding apparatus comprises a guide rail arranged in a fixed manner relative to the patient positioning surface,
wherein the counterpart comprises a contour sensor that contacts the guide rail, and
wherein the guide rail has a rail contour that is configured to insert the insertion element into the recording unit and/or remove the insertion element from the recording unit.

8. The magnetic resonance apparatus of claim 7, wherein the contour sensor is an element configured to roll and/or slide on the guide rail.

9. The magnetic resonance apparatus of claim 7, wherein the insertion element is configured to follow a movement of the contour sensor on the guide rail, and
wherein the movement is determined by the rail contour of the guide rail and is transmitted to the insertion element via the contour sensor.

10. The magnetic resonance apparatus of claim 7, wherein the counterpart comprises a spring configured to press the contour sensor against the guide rail.

11. The magnetic resonance apparatus of claim 10, wherein the spring is a leaf spring.

12. The magnetic resonance apparatus of claim 7, wherein the contour sensor is arranged coaxially relative to the insertion element.

13. The magnetic resonance apparatus of claim 1, wherein the counterpart is mounted in a rotatable manner relative to the recording unit.

14. The magnetic resonance apparatus of claim 1, wherein the counterpart is mounted so as to be rotatable about an axis of rotation connected to the radio frequency coil.

15. The magnetic resonance apparatus of claim 1, wherein the counterpart comprises a rotatably mounted pivot lever, and
wherein an insertion element and a contour sensor are arranged on the pivot lever.

16. The magnetic resonance apparatus of claim 1, wherein the holding apparatus comprises a guide rail arranged in a fixed manner relative to the patient positioning surface,
wherein the counterpart comprises a contour sensor that contacts the guide rail, and
wherein the guide rail has a rail contour that is configured to insert an insertion element into the recording unit and/or remove the insertion element from the recording unit.

17. The magnetic resonance apparatus of claim 16, wherein the contour sensor is an element configured to roll and/or slide on the guide rail.

18. The magnetic resonance apparatus of claim 16, wherein the insertion element is configured to follow a movement of the contour sensor on the guide rail, and
wherein the movement is determined by the rail contour of the guide rail and is transmitted to the insertion element via the contour sensor.

19. The magnetic resonance apparatus of claim 16, wherein the counterpart comprises a spring configured to press the contour sensor against the guide rail.
